(19) United States
(12) Patent Application Publication
(10) Pub. No.: US 2026/0297045 A1
(43) Pub. Date: Oct. 1, 2026

(54) A CONTINUOUS PROCESS FOR PREPARING ETHYLENE OXIDE

(71) Applicant: BASF SE, Ludwigshafen am Rhein (DE)

(72) Inventors: Nicolas DUYCKAERTS, Ludwigshafen am Rhein (DE); Jonglack KIM, Ludwigshafen am Rhein (DE); Phil Jack HOLZMEISTER, Ludwigshafen am Rhein (DE); Andrey KARPOV, Ludwigshafen am Rhein (DE); Wolfgang FISCHER, Ludwigshafen am Rhein (DE); Christian WALSDORFF, Ludwigshafen am Rhein (DE); Shane GROSSER, Ludwigshafen am Rhein (DE); Catharina KNIEKE, Ludwigshafen am Rhein (DE); Mirko HAIDER, Ludwigshafen am Rhein (DE)

(21) Appl. No.: 19/489,376

(22) PCT Filed: May 31, 2024
(86) PCT No.: PCT/EP2024/065095

§ 371 Date: Dec. 2, 2025

(30) Foreign Application Priority Data
Jun. 2, 2023 (EP) 23177024.9

(51) Int. Cl. C07D 301/10; G16C 20/10; G16C 20/70

(52) U.S. Cl. CPC C07D 301/10; G16C 20/10; G16C 20/70

(57) ABSTRACT

The present invention relates to a continuous process for preparing ethylene oxide in the presence of an activated heterogeneous silver catalyst and a production unit for carrying out said process.

The present invention relates to a continuous process for preparing ethylene oxide in the presence of an activated heterogeneous silver catalyst and a production unit for carrying out said process.

In the production of ethylene oxide, ethylene and oxygen in inlet gas react over an Ag containing catalyst. In order to achieve optimum EO selectivity, therefore, high ethylene efficiency chloride compound, e.g. mostly ethylene chloride, as a reaction moderator (or modifier) is fed into the reaction medium. The optimum chloride moderator concentration varies widely, depending on the chlorinated compound, the catalyst, operation temperature and gas phase composition. For example, in EP 1 458 698 B1, a process for the epoxidation of an olefin is disclosed which comprises reacting a feed comprising the olefin, oxygen and a reaction modifier in the presence of a silver-based catalyst. This patent relates to the avoidance of unwanted variations in the selectivity when the feed composition changes by changing the concentration of the reaction modifier such that a relative quantity Q of the reaction modifier is maintained substantially at a constant level at a given temperature. Temperature dependence of such a relative quantity Q is disclosed in EP 1 458 699 B1.

EP 3 087 061 B1 describes a method for the epoxidation of an olefin. This method is based on an equation correlating the change in moderator concentration with the change in temperature to attempt to keep the highly selective catalyst operating at peak efficiency in the oxidation of ethylene to ethylene oxide.

EP 1 458 698 B1 discloses a process for EO production comprising reacting a feed comprising the olefin, oxygen and a reaction modifier in the presence of a silver-based catalyst. The process relates to the avoidance of unwanted variations in the selectivity when the feed composition changes by changing the concentration of the reaction modifier such that a relative quantity Q of the reaction modifier is maintained substantially at a constant level at a given temperature. As indicated in the present application, the equations proposed in the prior art are temperature dependent such that it would require the multiple calculations of the Q by an operator every time the temperature is changing in the system during the operating time of the process This appears to be also the case in EP 1 458 698 B1. EP 3 087 061 B1 discloses a method for the epoxidation of an olefin and the development of an equation correlating the change in moderator concentration with the change of temperature to keep the highly selective catalyst operating at peak efficiency in the oxidation of ethylene to ethylene oxide.

CN 110 357 837 A discloses a process for the epoxidation of ethylene, using a highly selective catalyst, wherein the process comprises starting the process at given concentrations “normal concentrations”, after a certain time (more than 60 hours), decreasing the ethylene concentration and the chlorine compound concentration and waiting for temperature stabilization for about the same amount of hours, then increasing the CO₂ concentration, and waiting for stabilization of the temperature and continuing the process for more than 60 hours and finally restoring the ethylene, chlorine compound and CO₂ concentration to “normal concentrations. It appears that such changes in combination with the use of a highly-selectivity catalyst permit to improve the selectivity of the reaction.

However, in order to maintain high performance of the catalyst and facilitate the processes, there is always a need for an improved method for preparing ethylene oxide in the presence of a heterogeneous catalyst.

Therefore, the present invention relates to a continuous process for preparing ethylene oxide in the presence of an activated heterogeneous silver catalyst, the process comprising feeding ethylene, at least one chlorinated moderator and at least one saturated hydrocarbon into an epoxidation reactor containing said catalyst, wherein the process further comprises
(a) defining a target range of the work rate of the catalyst;
(b) adjusting at least one of the ethylene feed rate and the reactor temperature to realize a work rate of the catalyst in the range defined according to (a);
(c) adjusting the reactor inlet concentration of the at least one chlorinated moderator to maintain a factor F in the range of from 0.7×Fₜ to 1.3×Fₜ, and preferably 0.8×Fₜ to 1.2×Fₜ, and more preferably 0.9×Fₜ to 1.1×Fₜ;
wherein the factor F is a function of
(i) the reactor inlet concentration of the at least one chlorinated moderator;
(ii) the reactor inlet concentration of ethylene;
(iii) the reactor inlet concentration of the at least one saturated hydrocarbon;
(iv) the reactor temperature;
wherein Fₜ is a target value of the factor F and defines a set of process conditions at which optimum ethylene oxide selectivity is achieved.

In the context of the present invention, the process conditions are those listed above in (i) to (iv).

In the context of the present invention and as opposed to the disclosure in the prior art, Fₜ, the temperature independent target value of the factor F, defines a set of process conditions at which optimum ethylene oxide selectivity is achieved. It is determined experimentally as detailed in the present application and from that point onward, it is used as an instrument to facilitate decision-making regarding chlorination. Indeed, according to (c), the reactor inlet concentration of the at least one chlorinated moderator is adjusted to maintain a factor F in the range of from 0.7×Fₜ to 1.3×Fₜ, and preferably 0.8×Fₜ to 1.2×Fₜ, and more preferably 0.9×Fₜ to 1.1×Fₜ. Contrary to what is disclosed in the prior art, the target value of F can be determined once and F can be maintained in the range of from 0.7×Fₜ to 1.3×Fₜ, and preferably 0.8×Fₜ to 1.2×Fₜ, and more preferably 0.9×Fₜ to 1.1×Fₜ by dosing the addition of the chlorinated moderator during the operation time of the process. This permits to allow complete automation of the chlorination dosage as no recalculation of the factor would be necessary, even when the reactor temperature is increasing. This even permits the complete automation of the process in wide range of operation condition, such as reactor temperature.

Preferably, the target value Fₜ and the factor F are determined based on data obtained from one or more testing units and/or one or more production plants.

Most preferably, F is in the range of from 0.95×Fₜ to 1.05×Fₜ.

$$Preferably , the factor F = {f}_{1} ( c ) \times {f}_{2} ( {T}_{R} ) with$$

f₁(c) being a function of
(i) the reactor inlet concentration of the at least one chlorinated moderator,
(ii) the reactor inlet concentration of ethylene;
(iii) the reactor inlet concentration of the at least one saturated hydrocarbon;
f₂(T_{R}) being a function of
(iv) the reactor temperature T_{R}.

There is no insight in any of the prior art documents which would suggest the determination of a factor that needs to be maintained as constant as possible in order to have the combination of an optimum work rate combined with optimum ethylene oxide selectivity and that this can be optimized by adapting the moderator concentration based on its calculation from F.

Preferably, the at least one chlorinated moderator comprises ethyl chloride.

Preferably, the at least one chlorinated moderator further comprises one or more of vinyl chloride, methyl chloride and ethylene dichloride. More preferably, the at least one chlorinated moderator comprises ethyl chloride, vinyl chloride and methyl chloride.

$$Preferably , {f}_{1} ( c ) = [ c ( EC ) + c ( VC ) + 1 / 3 c ( MC ) ] / [ c ( e ) + {C}_{c} \times c ( a ) ] and {f}_{2} ( {T}_{R} ) = {C}_{T} - {T}_{R} ,$$

wherein

| | |
|---|---|
| c(EC) | is the concentration of ethyl chloride in an inlet gas stream G1 |
| | comprising ethylene, the at least one chlorinated moderator and |
| | the at least one saturated hydrocarbon fed into the epoxidation |
| | reactor; |
| c(VC) | is the concentration of vinyl chloride in G1; |
| c(MC) | is the concentration of methyl chloride in G1; |
| c(e) | is the concentration of ethylene in G1; |
| c(a) | is the concentration of the at least one saturated hydrocarbon in |
| | G1; |
| C_{C} | is constant during the operation time Δ t_{O} and has a value in the |
| | range of from 35 to 65, more preferably from 45 to 55; |
| C_{T} | is constant during Δ t_{O} and has a value in the range of from 285 |
| | to 340, more preferably from 305 to 320. |

C_{C} and C_{T} are constant values in the equations. These letters represent constant (numerical) values.

In the context of the present invention, the concentration of ethylene in G1 is the reactor inlet concentration of ethylene. Similarly, the concentration of the at least one saturated hydrocarbon in G1 is the reactor inlet concentration of the at least one saturated hydrocarbon.

Preferably, C_{C} is 48.

Preferably, C_{T} is 308.

$$Preferably , F = [ c ( EC ) + c ( VC ) + 1 / 3 c ( MC ) ] / [ c ( e ) + ( 45 to 55 ) \times c ( a ) ] \times ( ( 305 to 320 ) - {T}_{R} ) . More preferably , F = [ c ( EC ) + c ( VC ) + 1 / 3 c ( MC ) ] / [ c ( e ) + 48 \times c ( a ) ] \times ( 308 - {T}_{R} ) .$$

Preferably, the process further comprises periodically determining the factor F during the operation time Δt_{O} and adjusting the reactor inlet concentration of the at least one chlorinated moderator to maintain said factor F in the range of from 0.9×Fₜ to 1.1×Fₜ according to (c).

Preferably, periodically determining the factor F during the operation time Δt_{O} comprises periodically measuring the reactor temperature T_{R} and calculating F.

Preferably, periodically determining F during Δt_{O} comprises determining F during Δt_{O} every 1 min to 48 hours, more preferably every 1 min to 24 hours, more preferably every 2 min to 3 hours.

Preferably, the at least one saturated hydrocarbon is one or more of ethane, methane, propane, and cyclopropane, more preferably is one or more of ethane and methane.

Preferably, the reactor temperature T_{R} is in the range of from 180 to 350° C., more preferably in the range of from 190 to 300° C.

It should however be noted that the reactor temperature is varying during the operation time to adjust production capacity and/or to compensate the reduction in catalyst activity, as well known in the art by the skilled person.

Preferably, the pressure in the reactor is in the range of from 10 to 30 bar (abs), more preferably in the range of from 15 to 30 bar (abs).

Preferably, the activated catalyst comprises, in addition to silver, a support oxidic material and one or more promoters.

Preferably, the promoters comprise one or more of tungsten, rhenium, potassium, lithium and cesium.

Preferably, the support oxidic material preferably comprises alumina, more preferably α-alumina.

Preferably from 95 to 100 weight-%, more preferably from 97 to 100 weight-%, of the support oxidic material consists of alumina, more preferably α-alumina.

Preferably, the support oxidic material substantially consists of, or consists of, alumina, more preferably α-alumina

In the context of the present invention, the silver catalyst used in the present invention can be any silver containing catalyst known in the art. Preferably, the catalyst is as disclosed in EP 3 866 972 B1, which is incorporated herewith by reference.

Preferably, ethylene, the at least one chlorinated moderator and the at least one saturated hydrocarbon fed into the epoxidation reactor are comprised in an inlet gas composition, wherein ethylene is present in an amount in the range of from 1 to 50 mole-%, more preferably in the range of from 5 to 40 mole-%, based on the total moles of the inlet gas composition.

Preferably, the inlet gas composition further comprises up to 10 mole-% of oxygen, more preferably from 1 to 9 mole-%, based on the total moles of the inlet gas composition.

The inlet gas composition may further comprise carbon dioxide in an amount in the range of from 0 to 5 mole-% based on the total moles of the inlet gas composition.

Preferably, the inlet gas composition comprises the at least one saturated hydrocarbon in an amount in the range of from 30 to 70 mole-%, more preferably in the range of from 40 to 60 mole-%, based on the total moles of the inlet gas composition.

Preferably, the inlet gas composition has a content of the at least one chlorinated moderator in the range of from 0.01 to 50 ppm by volume (ppmv), more preferably in the range of from 0.1 to 30 ppmv, more preferably in the range of from 0.1 to 10 ppmv.

Preferably, (b) and (c) are performed one after the other or, preferably, (b) and (c) are performed simultaneously.

Preferably, one or more of (a), (b) and (c) are at least partially executed by using a computer or a computer network.

Preferably, one or more of the process steps as indicated above are performed by using a computer or a computer network, preferably by using a computer program.

The present invention further relates to a production unit for carrying out a process for preparing ethylene oxide according to the present invention, the production unit comprising
an epoxidation reactor comprising the activated heterogeneous silver catalyst;
a means for introducing ethylene into the reactor;
a means for introducing the at least one chlorinated moderator into the reactor;
a means for introducing the at least one saturated hydrocarbon into the reactor;
a means for removing ethylene oxide from the reactor;
a means for measuring the reactor temperature;
a means for measuring the reactor inlet concentration of the at least one chlorinated moderator;
a means for measuring the reactor inlet concentration of ethylene;
a means for measuring the reactor inlet concentration of the at least one saturated hydrocarbon;
a means for calculating the factor F.

The present invention further relates to a computer program product comprising a first computer-readable program code and a computer-readable medium, wherein the first computer-readable program code is recorded on the computer-readable medium and said first computer-readable program code is for instructing a data processing system of a computer system to execute calculations for a process for preparing ethylene oxide according to the present invention.

Preferably, the computer program product further comprises a second computer-readable program code, recorded on the computer-readable medium, for instructing the data processing system to control the process for preparing ethylene oxide according to the present invention.

The present invention further relates to a computer system comprising a data processing system and a computer program product according to the present invention.

Preferably, the computer system is configured to communicate with a means for measuring the epoxidation reactor temperature and with a means for introducing the at least one chlorinated moderator into the reactor to maintain a factor F in the range of from 0.7×Fₜ to 1.3×Fₜ, and preferably 0.8×Fₜ to 1.2×Fₜ, and more preferably 0.9×Fₜ to 1.1×Fₜ.

The present invention is further illustrated by the following set of embodiments and combinations of embodiments resulting from the dependencies and back-references as indicated. In particular, it is noted that in each instance where a range of embodiments is mentioned, for example in the context of a term such as “The process of any one of embodiments 1 to 4”, every embodiment in this range is meant to be explicitly disclosed for the skilled person, i.e. the wording of this term is to be understood by the skilled person as being synonymous to “The process of any one of embodiments 1, 2, 3 and 4”. Further, it is explicitly noted that the following set of embodiments represents a suitably structured part of the general description directed to preferred aspects of the present invention, and, thus, suitably supports, but does not represent the claims of the present invention.
1. A continuous process for preparing ethylene oxide in the presence of an activated heterogeneous silver catalyst, the process comprising feeding ethylene, at least one chlorinated moderator and at least one saturated hydrocarbon into an epoxidation reactor containing said catalyst, wherein the process further comprises
   (a) defining a target range of the work rate of the catalyst;
   (b) adjusting at least one of the ethylene feed rate and the reactor temperature to realize a work rate of the catalyst in the range defined according to (a);
   (c) adjusting the reactor inlet concentration of the at least one chlorinated moderator to maintain a factor F in the range of from 0.7×Fₜ to 1.3×Fₜ, and preferably 0.8×Fₜ to 1.2×Fₜ, and more preferably 0.9×Fₜ to 1.1×Fₜ;
   wherein the factor F is a function of
   (i) the reactor inlet concentration of the at least one chlorinated moderator;
   (ii) the reactor inlet concentration of ethylene;
   (iii) the reactor inlet concentration of the at least one saturated hydrocarbon;
   (iv) the reactor temperature;
   wherein Fₜ is a target value of the factor F and defines a set of process conditions at which optimum ethylene oxide selectivity is achieved.
2. The process of embodiment 1, wherein the target value Fₜ and the factor F are determined based on data obtained from one or more testing units and/or one or more production plants.
3. The process of embodiment 1 or 2, wherein F is in the range of from 0.95×Fₜ to 1.05×Fₜ.
4. The process of any one of embodiments 1 to 3, wherein the factor F=f₁(c)×f₂(T_{R}) with
   f₁(c) being a function of
   (i) the reactor inlet concentration of the at least one chlorinated moderator,
   (ii) the reactor inlet concentration of ethylene;
   (iii) the reactor inlet concentration of the at least one saturated hydrocarbon;
   f₂(T_{R}) being a function of
   (iv) the reactor temperature T_{R}.
5. The process of any one of embodiments 1 to 4, wherein the at least one chlorinated moderator comprises ethyl chloride.
6. The process of embodiment 5, wherein the at least one chlorinated moderator further comprises one or more of vinyl chloride, methyl chloride and ethylene dichloride, wherein more preferably the at least one chlorinated moderator comprises ethyl chloride, vinyl chloride and methyl chloride.
7. The process of embodiment 6, wherein

$${f}_{1} ( c ) = [ c ( EC ) + c ( VC ) + 1 / 3 c ( MC ) ] / [ c ( e ) + {C}_{c} \times c ( a ) ] \qquad ( 1 )$$

$${f}_{2} ( {T}_{R} ) = {C}_{T} - {T}_{R} \qquad ( 2 )$$

wherein
c(EC) is the concentration of ethyl chloride in an inlet gas stream G1 comprising ethylene, the at least one chlorinated moderator and the at least one saturated hydrocarbon fed into the epoxidation reactor;
c(VC) is the concentration of vinyl chloride in G1;
c(MC) is the concentration of methyl chloride in G1;
c(e) is the concentration of ethylene in G1;
c(a) is the concentration of the at least one saturated hydrocarbon in G1;
C_{C} is constant during the operation time Δt_{O} and has a value in the range of from 35 to 65, preferably from 45 to 55;
C_{T} is constant during Δt_{O} and has a value in the range of from 285 to 340, preferably from 305 to 320.
8. The process of any one of embodiments 1 to 7, further comprising periodically determining the factor F during the operation time Δt_{O} and adjusting the reactor inlet concentration of the at least one chlorinated moderator to maintain said factor F in the range of from 0.7×Fₜ to 1.3×Fₜ, and preferably 0.8×Fₜ to 1.2×Fₜ, and more preferably 0.9×Fₜ to 1.1×Fₜ according to (c).
9. The process of embodiment 8, wherein periodically determining the factor F during the operation time Δt_{O} comprises periodically measuring the reactor temperature T_{R} and calculating F.
10. The process of any one of embodiments 1 to 9, wherein the at least one saturated hydrocarbon is one or more of ethane, methane, propane, and cyclopropane, preferably is one or more of ethane and methane.
11. The process of any one of embodiments 1 to 10, wherein the reactor temperature T_{R} is in the range of from 180 to 350° C., preferably in the range of from 190 to 300° C.
12. The process of any one of embodiments 1 to 11, wherein the activated catalyst comprises, in addition to silver, a support oxidic material and one or more promoters, wherein the promoters preferably comprise one or more of tungsten, rhenium, potassium, lithium and cesium and wherein the support oxidic material preferably comprises alumina, more preferably α-alumina.
13. The process of any one of embodiments 1 to 12, wherein ethylene, the at least one chlorinated moderator and the at least one saturated hydrocarbon fed into the epoxidation reactor are comprised an inlet gas composition, wherein ethylene is present in an amount in the range of from 1 to 50 mole-%, preferably in the range of from 5 to 40 mole-%, based on the total moles of the inlet gas composition.
14. The process of embodiment 13, wherein the inlet gas composition further comprises up to 10 mole-% of oxygen, preferably from 1 to 9 mole-%, based on the total moles of the inlet gas composition.
15. The process of any one of embodiments 1 to 14, wherein one or more of (a), (b) and (c) are at least partially executed by using a computer or a computer network.
16. A production unit for carrying out a process for preparing ethylene oxide in the presence of an activated heterogeneous silver catalyst according to any one of embodiments 1 to 15, the production unit comprising
   an epoxidation reactor comprising the activated heterogeneous silver catalyst;
   a means for introducing ethylene into the reactor;
   a means for introducing the at least one chlorinated moderator into the reactor;
   a means for introducing the at least one saturated hydrocarbon into the reactor;
   a means for removing ethylene oxide from the reactor;
   a means for measuring the reactor temperature;
   a means for measuring the reactor inlet concentration of the at least one chlorinated moderator;
   a means for measuring the reactor inlet concentration of ethylene;
   a means for measuring the reactor inlet concentration of the at least one saturated hydrocarbon;
   a means for calculating the factor F.
17. A computer program product comprising a first computer-readable program code and a computer-readable medium, wherein the first computer-readable program code is recorded on the computer-readable medium and said first computer-readable program code is for instructing a data processing system of a computer system to execute calculations for a process for preparing ethylene oxide in the presence of an activated heterogeneous silver catalyst according to any one of embodiments 1 to 15.
18. The computer program product of embodiment 17, further comprising a second computer-readable program code, recorded on the computer-readable medium, for instructing the data processing system to control the process for preparing ethylene oxide in the presence of an activated heterogeneous silver catalyst according to any one of embodiments 1 to 15.
19. A computer system comprising a data processing system and a computer program product according to embodiment 17 or 18.
20. The computer system of embodiment 19, being configured to communicate with a means for measuring the epoxidation reactor temperature and with a means for introducing the at least one chlorinated moderator into the reactor to maintain a factor F in the range of from 0.7×Fₜ to 1.3×Fₜ, and preferably 0.8×Fₜ to 1.2×Fₜ, and more preferably 0.9×Fₜ to 1.1×Fₜ.

It is explicitly noted that the above set of embodiments represents a suitably structured part of the general description directed to preferred aspects of the present invention, and, thus, suitably supports, but does not represent the claims of the present invention.

In the context of the present invention, the term “operation time Δt_{O}” refers to the duration of the operation which starts when the target value Fₜ is first attained, namely when the set of process conditions (i) to (iv) is such that optimum ethylene oxide selectivity is achieved, until the end of the process. The operation phase of a process for preparing ethylene oxide starts after the completion of the start-up of the process which serves to finish the activation of the catalyst and attain the optimum conditions (selectivity/work rate).

In the context of the present invention, and as well-known in the art, the term “activated catalyst” refers to a catalyst which has been activated/conditioned during the start-up phase of the process for preparing ethylene oxide. The start-up phase begins at T=0 min of the process for preparing ethylene oxide and ends when the operation phase/time begins.

In the context of the present invention, it is noted that the term “moderator” can be used interchangeably with “promotor” or “activator” or “modifier”.

In the context of the present invention, the term “inlet gas” refers to the gas entering the epoxidation reactor. Hence, the term “inlet gas stream” refers to the (feed) gas stream entering the epoxidation reactor.

In the context of the present invention, the term “reactor temperature” refers to the average temperature in the reactor and in particular in the catalyst bed.

In the context of the present invention, a term “X is one or more of A, B and C”, wherein X is a given feature and each of A, B and C stands for specific realization of said feature, is to be understood as disclosing that X is either A, or B, or C, or A and B, or A and C, or B and C, or A and B and C. In this regard, it is noted that the skilled person is capable of transferring the above abstract term to a concrete example, e.g. where X is a chemical element and A, B and C are concrete elements such as Li, Na, and K, or X is a temperature and A, B and C are concrete temperatures such as 10° C., 20° C., and 30° C. In this regard, it is further noted that the skilled person is capable of extending the above term to less specific realizations of said feature, e.g. “X is one or more of A and B” disclosing that X is either A, or B, or A and B, or to more specific realizations of said feature, e.g. “X is one or more of A, B, C and D”, disclosing that X is either A, or B, or C, or D, or A and B, or A and C, or A and D, or B and C, or B and D, or C and D, or A and B and C, or A and B and D, or B and C and D, or A and B and C and D.

The present invention is further illustrated by the following examples.

### EXAMPLES

### Example 1

A process for preparing ethylene oxide has been run. Ethylene was subjected to catalytic gas phase oxidation using a molecular oxygen-containing gas over a catalyst bed comprising a heterogeneous silver catalyst, namely Catalyst 2.5 as described in Table 3 of the European patent EP 3 866 972 B1.

The epoxidation reaction was conducted in a vertically-placed test reactor constructed from stainless steel with an inner-diameter of 44 mm and a length of 12.80 m. The reactor was equipped with a thermocouple of an outer diameter of 8 mm positioned in the center of the reactor. The reactor temperature was regulated using pressurized water contained in the reactor mantel. Catalyst bed temperatures were measured using the thermocouple at five different positions equally distributed along the reactor length. All temperatures below refer to an average catalyst temperature of the five measurements. 16.3 kg of the heterogeneous silver catalyst was charged into the reactor so as to provide a catalyst bed with a height of 11.9 m. 0.65 m of inert ceramic balls were packed on top of the catalyst bed.

The catalyst was activated in a mixture of 55 to 60 Nm³/h of the feed reaction gas and 25 to 30 Nm³/h of nitrogen at an average catalyst temperature of 255 to 260° C. and a reactor outlet pressure of about 15 bar for about 36 hours. The feed reaction gas contained 35 to 40 vol.-% of ethylene, 6.5 to 7.5 vol.-% of oxygen, 0.4 to 0.8 vol.-% of carbon dioxide, 0.5 to 4 vol.-% of nitrogen, 0.1 to 0.3 vol.-% of ethane, 0.15 to 0.25 vol.-% of water, about 1 ppm of vinyl chloride, and methane as a balance gas. Additionally, 1.7 to 2.0 ppm of ethyl chloride were dosed into the feed during conditioning.

Subsequently, the catalyst temperature was decreased to about 235 to 240° C., the nitrogen flow was gradually decreased to 0 Nm³/h, and the reaction gas flow was gradually increased to adjust the GHSV to 4800 h⁻¹. Then, the temperature was adjusted to control a work rate at 280 kg (EO)/m³ (cat) h or an EO outlet concentration of 2.97 vol.-%. The amount of ethylene chloride (EC) dose, the moderator, was controlled to optimize concentration of chlorinated compounds in the inlet gas, thereby, to achieve the highest possible EO selectivity.

Composition of inlet and outlet gas were analyzed by gas chromatography equipped with a flame ionization detector (FID), which is for ethane and ethylene oxide, and three thermal conductivity detectors (TCD), which are for Ar, N₂, methane, CO₂, ethylene and water. Work rate, conversion of ethylene, and ethylene oxide selectivity were calculated as follows:

$$work rate [ {kg}_{EO} / {m}_{cat}^{3} / h ] = Volume of inlet gas [ {m}^{3} / h ] \cdot \frac{( Vol . - {\%}_{EO , out} \cdot SF - Vol . - {\%}_{EO , in} )}{100} \cdot \frac{0.044 [ kg / mol ]}{0.0224 [ {m}^{3} / mol ]} \cdot \frac{1000}{18.08 [ {m}_{cat}^{3} ]}$$

$$conversation of ethylene [ \% ] = 100 \cdot ( Vol . - {\%}_{ethylene , in} - Vol . - {\%}_{ethylene , out} \cdot SF ) / Vol . - {\%}_{ethylene , in}$$

$$selectivity of ethylene oxide [ \% ] = 100 \cdot ( Vol . - {\%}_{EO , out} \cdot SF - Vol . - {\%}_{EO , in} )$$

$$shrinkage factor ( SF ) = 100 / ( 100 + 0.5 \cdot ( Vol . - {\%}_{EO , out} - Vol . - {\%}_{EO , in} ) )$$

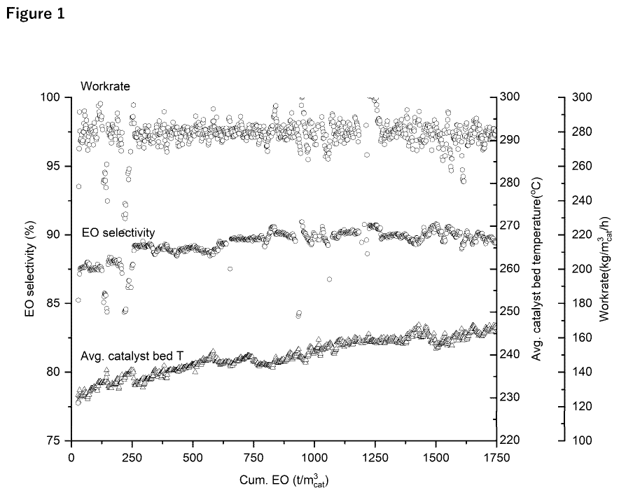

FIG. 1 displays work rate, EO selectivity and averaged catalyst bed temperature until currulative EO production (Curr. EO) of ca. 1750 t/m³_{cat}. EO process operated at work rate of ca. 280 kg (EO)/m³ (cat)/h. Temperature of catalyst bed was increased steadily to compensate deactivation of the catalyst.

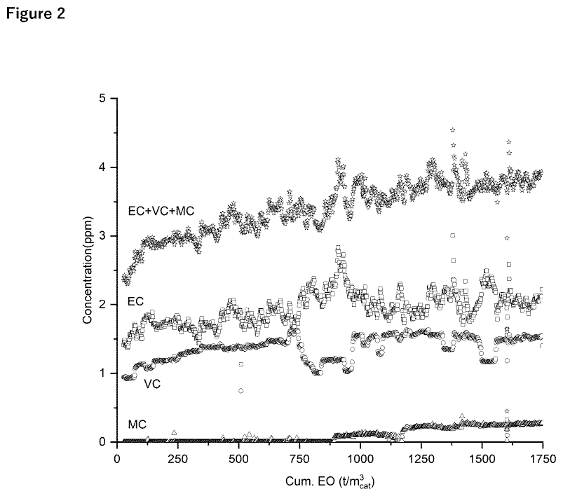

Optimal EO selectivity of 89-90% was achieved by adjustment of the moderator feed rate, namely ethylene chloride (EC) feed rate, to the recycling gas that also contained ethane, and other chloride compounds, e.g. vinyl chloride (VC) and methyl chloride (MC). Concentration of total chloride compounds, EC, MC and VC, in the inlet gas was increased for optimal EO selectivity time on stream, which is in line with lower chlorinating effect of chlorinated compounds at higher catalyst bed temperature (FIG. 2).

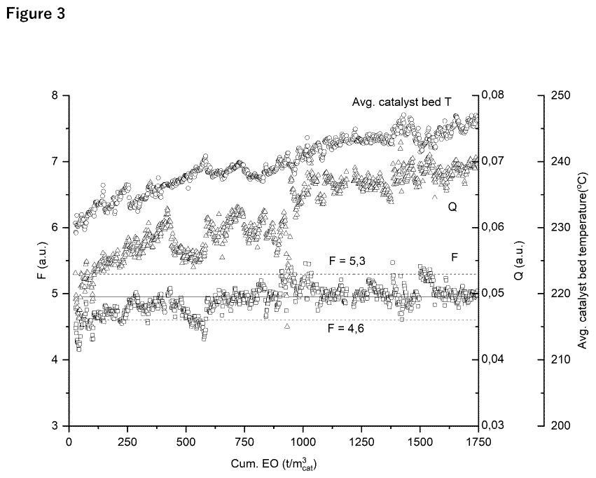

Based on the operation data of the EO process with optimal EO selectivity, a relative quantity Q of the reaction modifier as disclosed in EP 1 458 698 B1 and a factor of the invention F were calculated and plotted together against Curr. EO (FIG. 3). F=[c (EC)+c (VC)+1/3 c (MC)]/[c (e)+48×c (a)]×(308−T_{R}). Q and F were periodically determined during the operation time.

While the relative quantity Q was continuously increased by about 40% from 0.05 to 0.07, the factor F remained relatively constant in the range of 4.6 and 5.3 (4.9±0.4). When the factor F according to the present invention was reduced below 4.6, for example, at Curr. EO of 580 t/m³_{cat}, EO selectivity was decreased to below 89%. However, the optimal EO selectivity, over 89%, was recovered, when the factor F reached ca. 4.9 by adjusting ethylene chloride concentration in the inlet gas as well as catalyst bed temperature.

### Example 2

Ethylene oxide was produced by a selective oxidation of ethylene in a commercial plant, which was comprising of a process for production of ethylene oxide, purification of ethylene oxide and removal of carbon dioxide in recycling gas. Reaction feed gas was prepared by addition of ethylene, oxygen, methane and ethylene chloride moderator to the recycling gas.

Selective oxidation of ethylene to ethylene oxide is conducted in a typical tubular reactor, wherein a high selective catalyst comprised of Ag, Re, Li, Cs, K and alpha alumina support was filled. The reaction temperature was regulated by pressurized coolant/steam generated side of the reactor.

The catalyst was activated in a mixture of 55-65 vol.-% of the feed reaction gas balanced with nitrogen at a reactor temperature of 255 to 260° C. and a reactor outlet pressure of about 15 bar for about 36 hours. The feed reaction gas contained 25 to 40 vol.-% of ethylene, 4.0 to 7.5 vol.-% of oxygen, 0.4 to 0.8 vol.-% of carbon dioxide, 0.5 to 4 vol.-% of nitrogen, 0.1 to 0.3 vol.-% of ethane, 0.15 to 0.25 vol.-% of water, about 1 ppm of vinyl chloride, and methane as a balance gas. Additionally, 0.5 to 2.5 ppm of ethyl chloride were dosed into the feed during conditioning.

Subsequently, the reaction temperature was decreased to about 235 to 240° C., the nitrogen flow was gradually decreased to 0 Nm³/h, and the reaction gas flow was gradually increased to adjust the GHSV to 4800 h-1. Then, ethylene and O₂ concentration in the feed reaction gas, and temperature were adjusted to control a work rate at 280-300 kg (EO)/m3 (cat) h or an EO outlet concentration of 2.97-3.18 vol.-%. The amount of ethylene chloride (EC) dose, the moderator, was controlled by the factor F to optimize concentration of chlorinated compounds in the inlet gas, thereby, to achieve the highest possible EO selectivity.

Composition of feed gas and product gas were analyzed by a gas chromatography equipped with a flame ionization detector (FID), which is for ethane and ethylene oxide, and three thermal conductivity detectors (TCD), which are for Ar, N₂, methane, CO₂, ethylene and water. Work rate, conversion of ethylene, and ethylene oxide selectivity were calculated as described in Example 1.

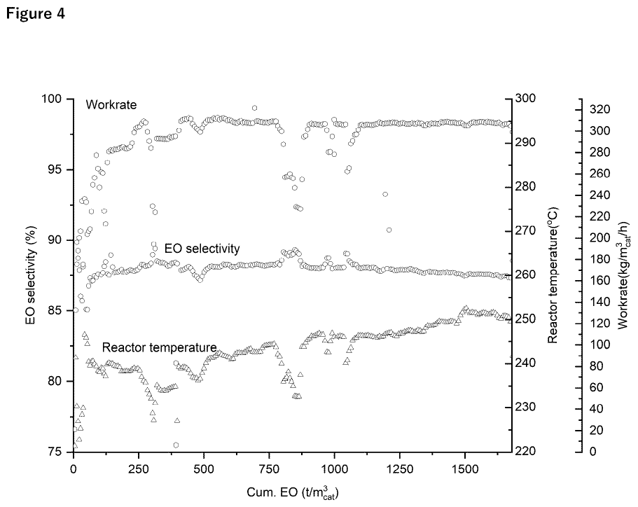

Work rate, EO selectivity and averaged catalyst bed temperature until currulative EO production (Curr.EO) of ca. 1700 t/m³_{cat} are displayed in FIG. 4. EO process operated at a work rate of 280 kg (EO)/m³ (cat)/h up to Curr.EO of 250 t/m³_{cat}. Afterwards, ethylene and O₂ concentration were increased stepwise to achieve a work rate of 300 kg (EO)/m³ (cat)/h. Temperature of catalyst bed was increased steadily to compensate deactivation of the catalyst.

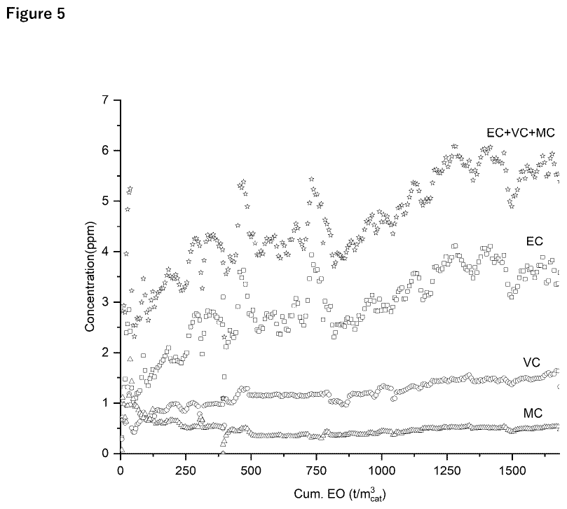

Optimal EO selectivity of 88-89% was achieved by adjustment of the moderator feed rate, namely ethylene chloride (EC) feed rate, to the recycling gas that also contained ethane, and other chloride compounds, e.g. vinyl chloride (VC) and methyl chloride (MC). Concentration of total chloride compounds, EC, MC and VC, in the feed gas was increased to achieve optimal EO selectivity time on stream, which is in line with lower chlorinating effect of chlorinated compounds at higher catalyst bed temperature (FIG. 6).

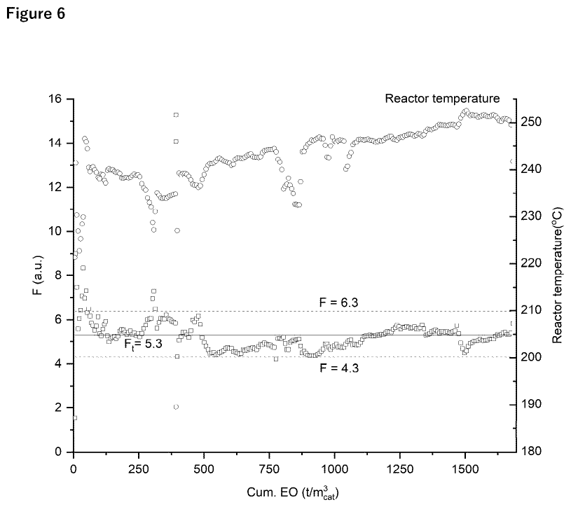

A factor of the invention F were calculated and plotted together with average catalyst bed temperature against Curr. EO (FIG. 7). F=[c(EC)+c(VC)+1/3 c (MC)]/[c (e)+48×c(a)]×(308−T_{R}). F were periodically determined during the operation time.

When the plant first reached a work rate of 280 kg (EO)/m³ (cat) h, the factor F was evaluated to be 5.3 at optimal EO selectivity. Afterwards, the rate of ethylene chloride dose was controlled by the factor F between 4.3 and 6.3 (5.3±1) at an increased work rate of 300 kg (EO)/m³ (cat)/h for operation of EO process with optimal EO selectivity.

Thus, thanks to the present invention, it is possible to determine the factor that needs to be calculated and maintained during the EO production such that a change in the moderator dosing be easily done as soon as the value of F is outside the acceptable ranges. Contrary to the factor in the art, the present invention proposes one which is temperature independent and permits a complete automation of the EO process.

### CITED LITERATURE

EP 1 458 699 B1
EP 1 458 698 B1
EP 3 087 061 B1
CN 110 357 837 A

1.-15. (canceled)

16. A continuous process for preparing ethylene oxide in the presence of an activated heterogeneous silver catalyst, the process comprising feeding ethylene, at least one chlorinated moderator and at least one saturated hydrocarbon into an epoxidation reactor containing said catalyst, wherein the process further comprises
(a) defining a target range of the work rate of the catalyst;
(b) adjusting at least one of the ethylene feed rate and the reactor temperature to realize a work rate of the catalyst in the range defined according to (a);
(c) adjusting the reactor inlet concentration of the at least one chlorinated moderator to maintain a factor F in the range of from 0.7×Fₜ to 1.3×F;
wherein the factor F is a function of
(i) the reactor inlet concentration of the at least one chlorinated moderator;
(ii) the reactor inlet concentration of ethylene;
(iii) the reactor inlet concentration of the at least one saturated hydrocarbon;
(iv) the reactor temperature;
wherein Fₜ is a target value of the factor F and defines a set of process conditions at which optimum ethylene oxide selectivity is achieved, namely when the set of process conditions (i) to (iv) is such that optimum ethylene oxide selectivity is achieved.

17. The process of claim 16, wherein the target value Fₜ and the factor F are determined based on data obtained from one or more testing units and/or one or more production plants.

18. The process of claim 16, wherein the at least one chlorinated moderator comprises ethyl chloride, the at least one chlorinated moderator further comprises one or more of vinyl chloride, methyl chloride and ethylene dichloride,
wherein the factor F=f₁(c)×f₂(T_{R}) with
f₁(c) being a function of
(i) the reactor inlet concentration of the at least one chlorinated moderator,
(ii) the reactor inlet concentration of ethylene;
(iii) the reactor inlet concentration of the at least one saturated hydrocarbon;
f₂(T_{R}) being a function of the reactor temperature T_{R},
and wherein
$${f}_{1} ( c ) = [ c ( EC ) + c ( VC ) + 1 / 3 c ( MC ) ] / [ c ( e ) + {C}_{c} \times c ( a ) ] \qquad ( 1 )$$
$${f}_{2} ( {T}_{R} ) = {C}_{T} - {T}_{R} , \qquad ( 2 )$$
wherein
c(EC) is the concentration of ethyl chloride in an inlet gas stream G1 comprising ethylene, the at least one chlorinated moderator and the at least one saturated hydrocarbon fed into the epoxidation reactor;
c(VC) is the concentration of vinyl chloride in G1;
c(MC) is the concentration of methyl chloride in G1;
c(e) is the concentration of ethylene in G1;
c(a) is the concentration of the at least one saturated hydrocarbon in G1;
C_{C} is constant during the operation time Δt_{O} and has a value in the range of from 35 to 65;
C_{T} is constant during Δt_{O} and has a value in the range of from 285 to 340.

19. The process of claim 16, wherein the factor F=f₁(c)×f₂(T_{R}) with f₁(c) being a function of
(i) the reactor inlet concentration of the at least one chlorinated moderator,
(ii) the reactor inlet concentration of ethylene;
(iii) the reactor inlet concentration of the at least one saturated hydrocarbon;
f₂(T_{R}) being a function of
(iv) the reactor temperature T_{R}, or
$$wherein the factor F = [ c ( EC ) + c ( VC ) + 1 / 3 c ( MC ) ] / [ c ( e ) + ( 45 to 55 ) \times c ( a ) ] \times ( ( 305 to 320 ) - {T}_{R} ) , or$$
$$wherein the factor F = [ c ( EC ) + c ( VC ) + 1 / 3 c ( MC ) ] / [ c ( e ) + 48 \times c ( a ) ] \times ( 308 - {T}_{R} ) .$$

20. The process of claim 19, wherein the at least one chlorinated moderator comprises ethyl chloride.

21. The process of claim 20, wherein the at least one chlorinated moderator further comprises one or more of vinyl chloride, methyl chloride and ethylene dichloride.

22. The process of claim 18, wherein the at least one chlorinated moderator comprises ethyl chloride, vinyl chloride and methyl chloride.

23. The process of claim 16, further comprising
periodically determining the factor F during the operation time Δt_{O} and adjusting the reactor inlet concentration of the at least one chlorinated moderator to maintain said factor F in the range of from 0.7×Fₜ to 1.3×F according to (c).

24. The process of claim 16, wherein the at least one saturated hydrocarbon is one or more of ethane, methane, propane, and cyclopropane.

25. The process of claim 16, wherein the reactor temperature T_{R} is in the range of from 180 to 350° C.

26. The process of claim 16, wherein ethylene, the at least one chlorinated moderator and the at least one saturated hydrocarbon fed into the epoxidation reactor are comprised an inlet gas composition, wherein ethylene is present in an amount in the range of from 1 to 50 mole-% based on the total moles of the inlet gas composition.

27. The process of claim 26, wherein the inlet gas composition further comprises up to 10 mole-% of oxygen based on the total moles of the inlet gas composition.

28. A production unit for carrying out a process for preparing ethylene oxide in the presence of an activated heterogeneous silver catalyst according to claim 16, the production unit comprising
an epoxidation reactor comprising the activated heterogeneous silver catalyst;
a means for introducing ethylene into the reactor;
a means for introducing the at least one chlorinated moderator into the reactor;
a means for introducing the at least one saturated hydrocarbon into the reactor;
a means for removing ethylene oxide from the reactor;
a means for measuring the reactor temperature;
a means for measuring the reactor inlet concentration of the at least one chlorinated moderator;
a means for measuring the reactor inlet concentration of ethylene;
a means for measuring the reactor inlet concentration of the at least one saturated hydrocarbon;
a means for calculating the factor F.

29. A computer program product comprising a first computer-readable program code and a computer-readable medium, wherein the first computer-readable program code is recorded on the computer-readable medium and said first computer-readable program code is for instructing a data processing system of a computer system to execute calculations for a process for preparing ethylene oxide in the presence of an activated heterogeneous silver catalyst according to claim 16.

30. A computer system comprising a data processing system and a computer program product according to claim 29.
